(19) United States
(12) Patent Application Publication
(10) Pub. No.: US 2021/0113414 A1
(43) Pub. Date: Apr. 22, 2021

(54) THERAPEUTIC ACTIVE-MOTION APPARATUS THAT INCREASES JOINT-ELECTRICITY FOR IMPROVING THE ACTIVE MOTION OF AFFECTED SHOULDER

(71) Applicant: SUE-MAY KANG, TAIPEI CITY (TW)

(72) Inventor: SUE-MAY KANG, TAIPEI CITY (TW)

(21) Appl. No.: 16/656,862

(22) Filed: Oct. 18, 2019

(51) Int. Cl. A61H 1/02; A61F 5/37; A61F 5/01; A63B 21/28; A63B 21/00

(52) U.S. Cl. CPC A61H 1/0281; A61F 5/3761; A61F 5/0118; A63B 21/285; A63B 21/00185; A61H 2201/1638; A63B 21/4035; A61H 2201/1276; A61H 2201/1207; A61H 2201/5025; A61H 2201/14; A63B 21/4017

(57) ABSTRACT

The present invention provides a therapeutic active-motion apparatus that increases Joint-Electricity for improving the active motion of affected shoulder. It is comprised of a straight-arm section, one or more than one arm-fixing strap, a hand-position section, and one or more than one force-applying-interface section, for applying a force toward the affected shoulder joint during the training of active motion to actualize the therapeutic mechanism based on Joint-Electricity Theory, and its application theory “Using the Favorable Component of Joint-Electricity-Generation as the favorable component in motion therapy”. The affected shoulders those with limitation in range of motion and shoulder pains use the present invention can achieve the effects including at least the increase in range of motion that is measurable, and reduction in the shoulder pains.

### FIELD OF THE INVENTION

The present invention is a therapeutic active-motion apparatus that increases Joint-Electricity for improving the active motion of the affected shoulders those defined as the shoulders involving the limitation in range of motion and pain. It is comprised of a straight-arm section, one or more than one arm-fixing strap, a hand-position section, and one or more than one force-applying-interface section, for applying a force toward the affected shoulder joint during the training of active motion to actualize the therapeutic mechanism based on Joint-Electricity Theory, and its application theory “Using the Favorable Component of Joint-Electricity-Generation as the favorable component in motion therapy”, both created by the present inventor. In the embodiments, the said force toward shoulder joint can be applied onto the said force-applying-interface section either by a hand, or, by an electrically operated pulling force. The goals of the therapeutic active-motion apparatus that increases Joint-Electricity for improving the active motion of affected shoulder of the present invention at least include the decrease in the pains and increase in the range of active motion of the affected shoulders.

### BACKGROUND OF THE INVENTION

Shoulder problems those with limitation in range of motion accompanied with shoulder pain (called affected shoulder in the present invention) are the problems those at nowadays still difficult to remedy. Within them, frozen shoulder, even with intact nervous system, is a typical one of the said affected shoulders, being disturbing the activities in daily living for a time period about two years. Thus, frozen shoulder is taken an example of affected shoulders as the goal of the present invention to afford therapeutic apparatus to effectively remedy. Frozen shoulder is also called “the shoulder in fifty” (in Taiwan), or, adhesive capsulitis of shoulder joint, or, idiopathic stiff shoulder. The main symptoms include shoulder pain combining difficulties in active motion, such as feeling weakness of shoulder muscles during motion, and limitation in range of motion. The complaints those frequently heard include cannot elevate the arm (flex the shoulder) to normal range, cannot handle the button(s) that at back, and, those severer even cannot comb the own hair, or, scratch the own back with the affected arm. The causing reasons of frozen shoulder have been attributed in various causes. Those described in previous literatures include (1) adhesive capsulitis of shoulder joint, and (2) fibrotic change in some of the shoulder tissues with unknown reason, and has been called “idiopathic stiff shoulder”. (This indicates that frozen shoulder is not originated from primary capsulitis, and when it is manifested in capsulitis, which is secondary). The main treating methods nowadays are first shoulder injection, and then motion therapies, such as physiotherapies, occupational Therapies, and home exercises. Yet, the general periods for significant improvement in their symptoms always exceed the patience of the patients, so that some patients have asked for capsular release, (or called release of scapulohumeral motion interface), or, mobilization with high intensity stretch. Despite that the said two methods have been reported improving the motion range faster than conventional exercise-program, they have also been reported having high recurrence rate in the previous literatures. Almost all the patients feel hard to receive a repeated surgery for capsular release, or, the said mobilization (that requiring anesthesia).

It is manifested that affected shoulders, including frozen shoulder, at nowadays still have not any satisfied treating method, nor, any effectively therapeutic active motion apparatus in the market.

### SUMMARY OF THE INVENTION

The present invention is a therapeutic active-motion apparatus that increases Joint-Electricity for improving the active motion of affected shoulder. It is invented based on Joint-Electricity Theory, and its application theory “Using the Favorable Component for Joint-Electricity-Generation as the favorable component in motion therapy”, both created by the present inventor. The primary objective of the present invention is to provide a therapeutic apparatus for managing the shoulder problems those related to limitation in range of shoulder motion accompanied with shoulder pain, such as frozen shoulder or other shoulder problems than frozen shoulder. Wherein, the said therapeutic active-motion apparatus of the present invention includes a unique structure that make possible for applying a force toward the affected shoulder joint to increase the generation of Joint-Electricity of the should joint so as to achieve an effect of improving the range of motion and reducing the shoulder pain.

The theoretical basis of the present invention is Joint-Electricity Theory that created by the present inventor, Sue-May Kang. The contents of this theory that related to the present invention include at least the following: (1). Joint-Electricity is generated through that the normal joint-cartilage has the property of piezoelectricity, and it can be always subjected to the normal dynamic force (that within its effective range) in the joint. On the contrary, if the said dynamic force is too large that exceeds its effective range, the said cartilage cannot generate Joint-Electricity. On the other hand, if the said dynamic force is too small with any reason, the generated Joint-Electricity will be not enough. (2). (2). The joint-structure tissues (capsule, ligaments, and tendons) and the related muscles of the said joint (those connecting to, or, overlapping the said joint) are called “the related tissue of the said joint”, or “the neighboring tissue of the said joint”, which require the “Joint-Electricity-Nurturing effect” from the Joint-Electricity that generated from the said joint. To describe in more details: the joint-Electricity that generated from the anterior part of the said joint joint-Electricity-Nurtures the anterior part of the said related tissue of the said joint, and vise-versa. The function of the said Nurturing includes at least maintaining the tissue-lengths and tones in normal, and getting ready for the ongoing muscular contraction. If the said Nurturing effect for the said part of the related tissue has been lacked or inadequate, there should be pains, shortening, and/or non-adequate tone of the tissues, decrease in the ranges of the active and passive motions, decrease in the muscular strength, and even fibrotic change occurred in the said part of the related tissue of the said joint in the severest conditions. (3) Further, if any part of the joint has the problem of inadequate generation of Joint-Electricity, the resulted inadequate Joint-Electricity-Nurturing of its related tissues would, in turn, result in the decrease in the tone, or, length of them, and consequently the alteration in the dynamic force that would cause further decrease in the generation of the Joint-Electricity from the said part of the joint. This vicious cycle makes the further decreasing in the Joint-Electricity-Nurturing on the said related tissues, and consequent the severer problems resulted from inadequate Joint-Electricity-Nurturing on the said tissues. (4). Active motion requires muscular contraction, and based on Joint-Electricity Theory, muscular contraction requires first being well Joint-Electricity-Nurtured, so that the subjects those stated in (3) should first be supplemented Joint-Electricity before conducting any active motion, or using an adequate apparatus that can increasing Joint-Electricity (such as the present invention) during the training of the said active motion, in order to conduct the active motion in the condition that the related muscles are enough Joint-Electricity-Nurtured. (5). The “related theory” that originated from the said JOINT-ELECTRICITY THEORY, called “Favorable-component for Joint-Electricity-Generation”, which includes that “when the dynamic force at the same site of a joint is increased to an amount that doesn't exceed its effective range, the said larger amount of the dynamic force can increase the generation of the Joint-Electricity in the said part of the joint, thus increases the extent of Joint-Electricity-Nurturing of the related muscles of the said site of the said joint, and consequent the increase in the muscular strength of the said muscles during a forceful active motion. The said theory can be used during motion therapy, and is called “Using the Favorable-Component for Joint-Electricity-Generation as the favorable component in motion therapy”. This theory includes the following. For the subjects having defect in Joint-Electricity generation due to too small in the dynamic force in at least one part of the joint, and consequent motion deficits, usage of the methods based on the said theory, such as using manual method, or therapeutic apparatus to offer the increase of the dynamic force in the said site of the joint that does not exceed its effective range can increase the Joint-Electricity generated from the said site of the joint, and consequently increases the effect of the said motion therapy, including at least the increase in motion ability (which includes at least the increase in the range of motion).

Thus, to achieve the objective of managing effectively on the affected shoulders those stated above, the present invention provides a “therapeutic active-motion apparatus that increases Joint-Electricity for improving active motion of affected shoulder”, which is comprised of a straight-arm section, one or more than one arm-fixing strap, a hand-position section, and one or more than one force-applying-interface section.

The said straight-arm section has longitudinal rigidity and a length that is sufficient to receive and support an affected arm of a user in a straight position in order to provide an effect of keeping the affected arm in the straight position during the usage of the present invention.

The said arm-fixing strap can be fixed to, or additionally attached to, the straight-arm-section at least on its shoulder-end and the site corresponding to the elbow, to have an effect of firmly fixing the straight-arm section to the straight positioned arm of the user.

The said hand-position section has one-end connected to the hand-end of the said straight-arm section, and has a shape of being readily gripped by, or easily fixed to, the hand of the user at the location correspond to the hand of the user on the extension of axial line of the said straight-arm section.

The said force-applying-interface section, each of them is arranged on the straight-arm section at the location corresponding to one of the required directions of the motion of the affected arm. It has a shape for easily pulling the entire affected arm that is fixed in the therapeutic active-motion apparatus of the present invention for applying a force toward the affected shoulder joint by a hand of the therapist of the said user, or the contralateral hand of the user. Wherein, one of the said force-applying-interface sections that on the side that the arm moves toward is selected to be used for applying the force toward the affected shoulder joint during the active motion that on the said direction.

In an embodiment of “therapeutic active-motion apparatus that increases Joint-Electricity for improving active motion of affected shoulder” of the present invention that stated above, a gliding section is further included, which is fixed to, or, connected by a gliding-section-connecting device to, the distal end of the said hand-position section, or the said straight-arm section. Wherein, the said gliding section has a distal end in low friction for easing the gliding on a fixing surface.

In an embodiment of “therapeutic active-motion apparatus that increases Joint-Electricity for improving active motion of affected shoulder” of the present invention, it further includes an electrically-operated-pulling section to offer the pulling force for substituting the force applied by a hand during the active motion of the user that stated in [0006].

The said electrically-operated-pulling section is comprised of an electrically-operated-pulling unit, and a shoulder-chest-strap that fits to the target shoulder for locating the said electrically-operated-pulling unit on the top-shoulder area of the affected shoulder. Thus, the said electrically-operated-pulling section is also called “electrically-operated-pulling-unit-that-situated-on-top-of-shoulder”.

The said electrically-operated-pulling unit is comprised of an object-strap, an electric machine, a support-post, a force-wire-supporting-point, and a force-wire.

The said object-strap is made of a bendable material, and has a connecting device at its underside.

The said electric machine has the function of pulling the force-wire, and is securely fixed to the top surface of the said object-strap. Wherein, the pulling force generated by the said electric machine can be controlled by a force-level-adjusting switch, and anon/off switch. Wherein, the said force-level-adjusting switch is for pre-setting the force to be applied during the instant active motion of the user immediately before the performance of it. The said on/off switch is for the control of the beginning/stop of the said force generated by the said electric machine during the active-motion of the affected shoulder.

The said support-post has a height that is adjustable, and is securely fixed to the top surface of the said object-strap on the right side, and, left side, respectively, of the side having the opening for the said force-wire to run out of the said electric machine, thus, the said electrically-operated-pulling unit has the types for right-side usage, and, left-side usage, respectively.

The said force-wire-supporting-point allows the said force-wire to go through, and change its direction, with low friction. The said force-wire-supporting-point is comprised of a circle in smooth surface, and a thin and short post that is fixed under it, wherein, the said thin and short post is fixed to the top of the said support-post in a way that the said circle can be rotated in order to decrease any friction when the force-wire is pulled through it.

The said force-wire has one-end connecting to the said electric machine, and the other-end going through the said force-wire-supporting-point, and then connecting to the one force-applying-interface section that on the side that the arm will move toward in the ongoing training session, and is pulled by the said electric machine to apply a force toward the affected shoulder joint.

The said shoulder-chest-strap is designed to be that can fit to the shoulder-chest area of the affected side of the individualized user, and has a connecting device on the area corresponding to the top area of the affected shoulder for connecting with the counterpart connecting device that under the object-strap, in order to situate the said electrically-operated-pulling unit on the top of the affected shoulder of the user before using. The said shoulder-chest-strap is comprised of a chest-strap and a shoulder-strap.

The said chest-strap has a length-adjusting device to adjust its length for matching the circumference of the chest of the user, and has a connecting device set at one-end, and two connecting device s respectively set at the position corresponding to the central portion of the chest of the user, and the other-end of the said chest-strap.

The said shoulder-strap is secured, at an angle that is about 25 degrees, to the said chest-strap at the position corresponding to the central portion of the back of the user, and then it runs over the top of the affected shoulder and arrives at and is connected to the said connecting device that at the central portion of the chest of the user that on the said chest-strap. Further, on the said shoulder-strap, at the area corresponding to the shoulder-top of the user, there is a connecting device of the type that is the counterpart of that fixed at the underside of the object-strap.

As such, the patients those with affected shoulders can use the said “therapeutic active-motion apparatus that increases Joint-Electricity for improving active motion of affected shoulder” of the present invention, either by a hand, or, by the said electrically-operated-pulling section, to apply the required force toward the affected shoulder during the training of active motion for achieving the goals including immediate perception of pain reduction, and the increase of range of motion that is measurable. The present invention is applicable not only to frozen shoulder, but also the shoulder problems other than frozen shoulder for promoting their faster improvement on active motion during the process of removing the major cause of disability or disease.

For better understanding and appreciation of objectives, technical features, and effectiveness of the present invention, a detailed description will be provided below with reference to embodiments of the present invention and drawings thereof.

### BRIEF DESCRIPTION OF THE DRAWINGS

FIG. 1 is a back schematic view of an embodiment of a therapeutic active-motion apparatus that increases Joint-Electricity for improving the active motion of affected shoulder of the present invention;

FIG. 2 is a side schematic view of the embodiment that shown in FIG. 1 (an example in which only one force-applying-interface section is provided);

FIG. 3 is a side schematic view of using the embodiment that shown in FIG. 1 (an example in which more than one force-applying-interface section is provided), wherein, a force is applied toward the affected shoulder joint by a hand;

FIG. 4 is a front schematic view of using another embodiment of the present invention, which has included an electrically-operated-pulling section, wherein, the said force-applying-interface section is connected to the force-wire of an electrically-operated-pulling unit to apply the force toward the affected shoulder joint; and

FIG. 5 is a schematic view illustrating a detailed structure of an electrically-operated-pulling unit.

### DETAILED DESCRIPTION OF THE PREFERRED EMBODIMENTS

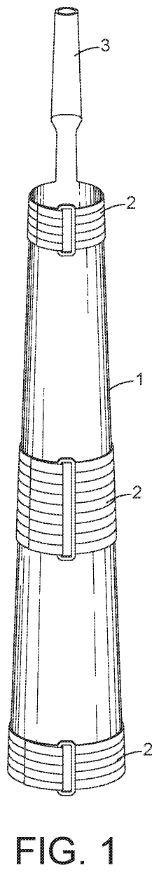

The embodiments of the present invention will be described below with reference to FIGS. 1-5. The description is not provided for constraining the ways of embodying the present invention, and is provided for explaining one or some ways of the embodiment of the present invention.

The present invention provides a therapeutic active-motion apparatus that increases Joint-Electricity for improving the active motion of affected shoulder, which is invented based on Joint-Electricity Theory, and its application theory “Using the Favorable Component for Joint-Electricity-Generation as the favorable component in motion therapy”, both created by the present inventor.

Firstly, referring to FIGS. 1-3, the present invention provides a therapeutic active-motion apparatus that increases Joint-Electricity for improving the active motion of affected shoulder, which is comprised of a straight-arm section 1, one or more than one arm-fixing strap 2, a hand-position section 3, and one or more than one force-applying-interface section 4.

The said straight-arm section 1 has longitudinal rigidity, and a length that is sufficient to receive and support an affected arm of a user in a straight position in order to provide an effect of keeping the affected arm in the straight position during the usage of the present invention. The said straight-arm section 1 can be made of, for example, the sheet-form material (as shown in FIG. 1). Further, the said straight-arm 1 may have one or multiple ventilation holes over the said sheet-form structure, wherein the said ventilation holes can be the openings of any shape on the sheet-form structure. However, the form of the material that made of the straight-arm section is not limited to that stated above, and are known to the art.

The said one or more than one arm-fixing strap 2 can be securely fixed to or additionally attached to the straight-arm section 1 at least on its shoulder-end and the site corresponding to the elbow to have an effect of firmly fixing the straight-positioned arm of the user to the said straight-arm section 1. However, the present invention has not limited any specific way of making the said arm-fixing strap 2, or, any specific part of straight-arm section 1 to which the said arm-fixing strap 2 is applied for individualized users, because these are considered known to the art.

The said hand-position section 3 has one-end connected to the hand-end of the said straight-arm section 1, and has a shape of being readily gripped by, or fixed on, the hand of the user at the location correspond to the hand of the user on the extension of axial line of the said straight-arm section 1. The said shape can be a configuration of a stick for holding as shown in FIGS. 1-3, or other shapes those are easily held for fixing a hand position, such as the various handles of umbrella, or, crutch, or, cane, or, walking stick. However, the shapes of the hand-position section are those known to the art.

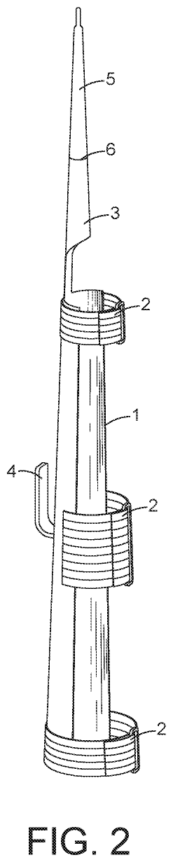

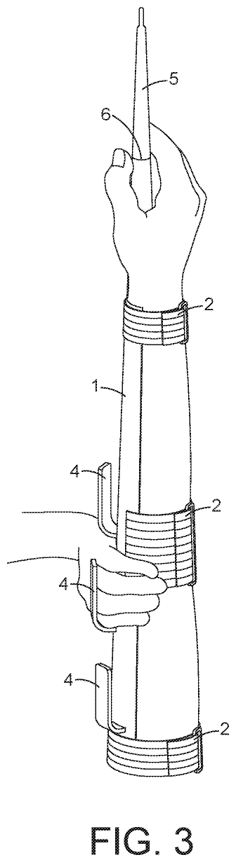

The said one or more than one force-applying-interface section 4, each of them is arranged on the straight-arm section 1 at the location corresponding to one of the required directions of the motion of the affected arm. It has a shape for easily pulling the entire affected arm that is fixed in the therapeutic active-motion apparatus of the present invention in order to easily correctly apply a force toward the affected shoulder joint by an a hand of the therapist of the user, or the contralateral hand of the user. Wherein, one said force-applying-interface section 4 that on the side that the arm moves toward is used for applying the force toward the affected shoulder joint during the active motion that on the said direction. The shapes of the said force-applying-interface section 4 can be an aperture formed in the straight-arm section 1, or, a circular ring or a hook raised from the straight-arm section 1, or, additionally attached thereto, a recess having an opening facing away from the shoulder joint (as shown in FIGS. 2 and 3). However, the shapes of the said force-applying-interface are those known to the art.

The embodiments shown are for managing the shoulder problems those with the motion difficulties in shoulder flexion, shoulder abduction, or shoulder horizontal abduction. In case that the number of the said force-applying-interface section 4 is greater than one, they could be arranged on the said straight-arm section 1 at positions corresponding to a front side, a lateral side, and a combined angular position thereof of the arm (as shown in FIG. 3). This allows the selection of the one thereof at the side the arm moves toward during the performance of the arm motion in the said direction. This makes possible for applying a force on correct direction toward the shoulder joint during the active motion of the arm in the said direction. In case that the number of the force-applying-interface section 4 is only one, it can be arranged on the straight-arm section 1 at the position corresponding to a front, or lateral side of the arm (as shown in FIG. 2), or a combined angular position thereof, for the training on either direction, for individual purchased. For managing the motion difficulties involved other directions, the sites of the said force-applying-interface can be varied from those stated above, and that the said sites are those easily figured out by the art.

In an embodiment of the therapeutic active-motion apparatus that increases Joint-Electricity through an electrically-operated-pulling-force to increase the active motion of affected shoulder of the present invention, a gliding section 5 is further included. The said gliding section can be fixed to, or connected by a gliding-section-connecting device 6 (such as screw, but does not limit to it) to, the distal end of the said hand-position section 3 (as shown in FIGS. 2 and 3), or the said straight-arm section. Wherein, the said gliding section 5 has its distal end in low friction, such as rounded, smooth, and small. Yet, the configuration of the end of the said gliding section for making it in low friction is that known to the art. The said gliding section 5 can glide on a fixing surface, especially on the wall, during the initial training stage, or the stage that easily fatigues, for easing the performance of the target active motion. When it is not required, it can be removed.

The using methods of the said therapeutic active-motion apparatus that increases Joint-Electricity for improving the active motion of affected shoulder of the present invention are described in the following.

Firstly, the affected arm of a user is positioned in a straight position on the said straight-arm section 1, is fastened by the arm-fixing strap 2 for fixing the said straight arm to the said straight-arm section 1, and allows the hand of the affected arm to hold, or be fixed to, the hand-position section 3.

Second, the direction of the active motion to be performed in the instant period is determined, and the one force-applying-interface section that on the side that the arm moves toward is used to operate applying the force toward the affected shoulder joint by a hand of the therapist of the user during the active motion in the said direction in order to actualize the mechanism of increasing generation of Joint-Electricity (as shown in FIG. 3).

Further, the force applied is not large, being cautioned does not exceed the effective range of the dynamic force within the relevant part of the said shoulder joint, which the motion-therapist that has been well trained for using the said present invention, and experienced on managing the subjects in various injured-severity, recovery stages, and weight of the said arm would know how to adjust its individualized magnitudes for the individualized user along the therapy sessions. Only when the user gets skilled in performing the operation by oneself, the user can use the own hand of the healthy-side to apply the force toward the affected shoulder joint under supervision in clinics, and afterward at home.

The cautions during the usage of the therapeutic active-motion apparatus that increases Joint-Electricity for improving the active motion of affected shoulder of the present invention are stated in the following.

The said therapeutic active-motion apparatus of the present invention can be used to benefit to the patients with the affected shoulders involving the limitation in range of active motion accompanied with pain. Using the therapeutic active-motion apparatus of the present invention, or learning to use it should be in the motion-therapy that conducted by a motion-therapist who is an occupational therapist or a physiotherapist that has taken courses and training of the present invention.

Further, since the shoulder joint, in fact, involves multiple joints, arm elevation, especially between 120 to 180 degrees, require the coordination of motions in axial direction and rotating direction, and thus, the motion-therapist must provide teaching, at proper timing, regarding the coordination between the two motions. Thus, arm-flinging is prohibited. The most of all, the training of active motion with the said present invention, in general, includes the active exercise of the affected arm in an attempt to increase the angle of the active motion, especially shoulder flexion, shoulder abduction, shoulder horizontal abduction, and the motion of the combined angles thereof. In consideration of the unique anatomic structure of the shoulder joint, and that arm elevation, once exceeding 120 degrees, is accompanied with the gradual increase in external rotation of the arm, so that, at the beginning of the said training, the affected arm may take a posture of maximal external rotation as being fixed in the straight-arm section 1 for carrying out the adequate motions stated above. Further, before use, the said straight-arm section 1 must be correct and proper fixed to the straight positioned arm of the affected side of the user. If operation starts without proper fixing, it would be hard to control the direction of the force applied.

It is also cautioned that arm-elevation conducted in different directions requires selecting and using the one force-applying-interface section 4 on the side the arm moving toward, respectively. When the active motion involves shoulder flexion, the force-applying-interface section 4 that is located at the front side of the arm may be used. When the active motion involves shoulder horizontal abduction, the one that is located at the lateral side may be used. When the active motion involves a combined angle arm-raising, the one located at the side the arm moves toward may be used (as shown in FIG. 3). Further, when the active motion involves shoulder abduction, the affected arm can be, first, positioned in maximal external rotation in the straight-arm section 1, and use the one that located in the side that the arm moves toward (which is actually the one located at the front side of the arm, if a maximal external rotation has been achieved).

It should be emphasized that the force applied by the usage of the present invention is not in the direction of the active motion of the arm, but only that toward the affected shoulder joint. Note that it is also prohibited to use the present invention to apply the said force toward a healthy joint due to which would make the dynamic force within the said joint exceed its effective range, and consequent a bad result.

The therapeutic active-motion apparatus that increases Joint-Electricity for improving the active motion of affected shoulder of the present invention provides the said force-applying-interface section 4 located on the side that the affected arm moving toward for applying the force toward the affected shoulder joint by a hand (of the motion-therapist of the user) during the active motion in the said direction, so as to actualize “a therapeutic mechanism of increasing Joint-Electricity-generation on the part (s) of the affected shoulder joint that relevant to the ongoing motion that following Joint-Electricity Theory, and its application theory “Using the favorable Component for Joint-Electricity-Generation as the favorable component in motion therapy”, and consequently achieve the goals at least of immediate perception of pain-reduction, and the increase in range of active motion that is measurable. Further, the theory of the present invention and a similar structure thereof may be applied to other joints. The application of the present invention to shoulder is just an example for illustration of the present invention.

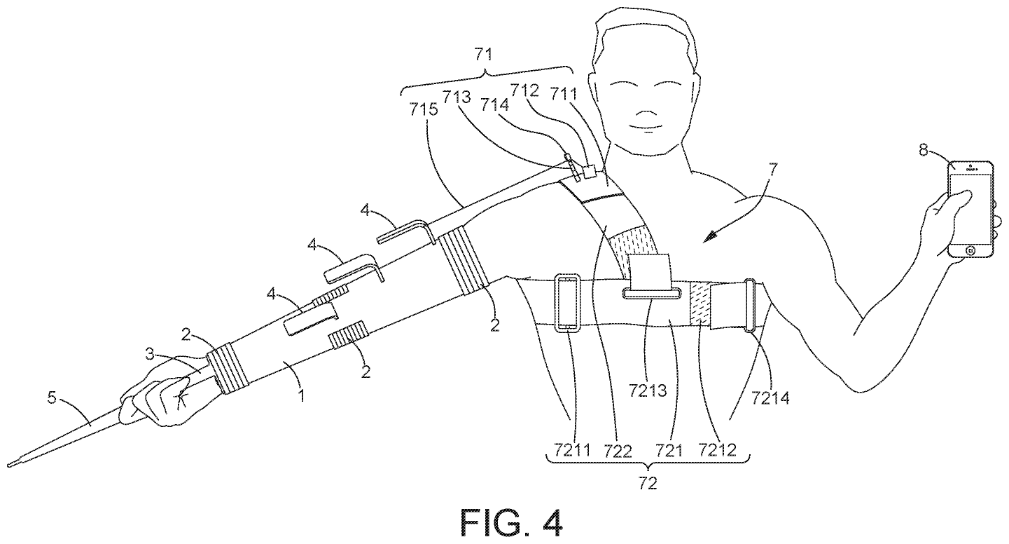

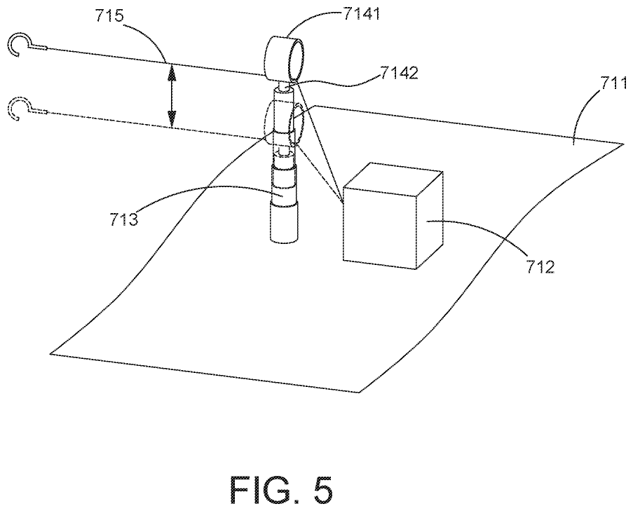

Referring to FIGS. 4-5, in addition to FIGS. 1-3, the present invention provides another embodiment that further includes an electrically-operated-pulling section 7 to be connected to the said force-applying-interface section 4 to offer the required pulling force (as shown in FIG. 4) for substituting the force applied by a hand during the active motion of the user to actualize the therapeutic mechanism of increasing generation of Joint-Electricity. The said electrically-operated-pulling-section 7 is actually an electrically-operated-pulling unit 71 that is situated (fixed) on a shoulder-chest-strap 72 that can fit to the target shoulder, thus, is also called “electrically-operated-pulling-unit-that-situated-on-top-of-shoulder”.

Wherein, the said electrically-operated-pulling unit 71 is comprised of an object-strap 711, an electric machine 712, a support-post 713, a force-wire-supporting-point 714, and a force-wire 715.

The said object-strap 711 is made of a bendable material in order to be used in a curved surface. Further, there is a connecting device (such as a hook side, or a loop side, of a hook-and-loop fastener) at its underside for connecting itself (thus, the whole electrically-operated-pulling unit) to a site requires it during using. However, the connecting device s can be in various types, and are known to the art.

The said electric machine 712 has the function of pulling the force-wire 715, and is fixed to the top surface of the said object-strap 711. Wherein, the pulling force generated by the said electric machine 712 can be controlled by a force-level-adjusting switch, and an on/off switch. Wherein, the said force-level-adjusting switch is for pre-setting the force to be applied during the instant active motion of the user immediately before the performance of it. The said on/off switch is for the control of the beginning/stop of the said force generated by the said electric machine during the active-motion of the affected shoulder.

The said support-post 713 has a height that is adjustable, and is fixed to the top surface of the said object-strap 711 on the right side, and, left side, respectively, of the side having the opening for the said force-wire to run out of the said electric machine 712, thus, the said electrically-operated-pulling unit has right-side-usage type, and, left-side-usage type, respectively.

The said force-wire-supporting-point 714 allows the force-wire 715 to go through, and change its direction, with low friction. It is comprised of a circle in smooth surface 7141, and a thin and short post 7142 that is fixed under it, wherein, the said thin and short post 7142 is fixed to the top of the said support-post 713 in a way that the said circle in smooth surface 7141 can be rotated in order to decrease any friction when the force-wire 715 is pulled through it. Wherein, the methods for making the said circle in smooth surface, and the fixing method between the said circle in smooth surface 7141 and support-post 713, are those known to the art.

The said force-wire 715 has one-end to be connected to the said electric machine 712, and the other-end to go through the said force-wire-supporting-point 714 and then to connect to one force-applying-interface section 4 that on the side that the arm will move toward in the ongoing training session, and is to be pulled by the said electric machine 712 to apply a force toward the affected shoulder joint.

The said shoulder-chest-strap 72 is designed to be that can fit to the shoulder-chest area of the affected side of any individualized user, and has a connecting device on the area corresponding to the top area of the affected shoulder for connecting with the counterpart connecting device that under the object-strap, in order to situate the said electrically-operated-pulling unit 71 on the top of the affected shoulder of the user before using. Thus, the said electrically-operated-pulling section 7 that comprised of an electrically-operated-pulling unit 71 and a shoulder-chest-strap 72 is also called “electrically-operated-pulling-unit-that-is-situated-on-t op-of-shoulder”.

The said shoulder-chest-strap 72 is comprised of a chest-strap 721, and a shoulder-strap 722.

The said chest-strap 721 has a length-adjusting device 7211 to adjust its length for matching the circumference of the chest of the user, and has a first type connecting device 7212 (such as loop and hook of Velcro in continuation) set at one-end, and two of the second type connecting device s those in coordination to the said first type connecting device (such as rectangular D-ring) 7213, and, 7214, respectively, set at the position corresponding to the central portion of the chest of the said chest-strap, and, the other-end of the said chest-strap 721, respectively.

The said shoulder-strap 722 is fixed, at an angle that is about 25 degrees, to the said chest-strap 721 at the position corresponding to the central portion of the back of the user, and then it runs over the top of the affected shoulder, and arrives at, and is connected to, the said second type of connecting device 7213 (such as rectangular D-ring) that at the central portion of the chest on the said chest-strap 721. Further, the top side of the said shoulder-strap 722 has a connecting device on the area corresponding to the top-shoulder area of the affected shoulder. Wherein, the said connecting device is for connecting with the counterpart connecting device under the object-strap 711 in order to position the said electrically-operated-pulling unit 71 on the top of the affected shoulder of the user.

Wherein, the types of connecting device s, and the fixing method of the connecting device fixing to the required sites are those known to the art.

In an embodiment of the therapeutic active-motion apparatus that increases Joint-Electricity for improving active motion of affected shoulder that has further included an electrically-operated-pulling section (electrically-operated-pulling-unit-that-is-situated-on-t op-of-shoulder) further includes a remote control device 8 to remotely control the operation of the said electric machine 712. The said remote control device 8 includes at least a force-level-adjusting switch, and an on/off switch. The said remote control device 8 can be manually controlled, but is not limited to manual control. However, the inclusion of a remote control device for any electrically-operated device is known to the art.

In an embodiment of the therapeutic active-motion apparatus that increases Joint-Electricity for improving active motion of affected shoulder that has further included an electrically-operated-pulling section (electrically-operated-pulling-unit-that-is-situated-on-t op-of-shoulder) further includes a gliding section 5. The said gliding section 5 is connected by a gliding-section-connecting device 6 (such as screw, but does not limit to it) to the distal end of the said hand-position section 3, or, the said straight-arm section 1, wherein, the said gliding section 5 has the distal end in low friction, such as rounded, smooth, and small. Yet, the configuration of the end of the said gliding section for making it in low friction is that known to the art. The said gliding section 5 can glide on a fixing surface, especially on the wall, during the initial training stage, or the stage that easily fatigue, for easing the performance of the target active motion. When it is not required, it can be removed.

The using methods of the therapeutic active-motion apparatus that increases Joint-Electricity for improving the active motion of affected shoulder of the present invention that has further included an electrically-operated-pulling section 7 to offer the required force toward the affected shoulder joint are described in the following.

Firstly, the shoulder-chest-strap 72 that of the affected side is put on, in a neat and fit manner, on the shoulder-chest portion in affected side of a user by using the length adjusting device 7211, and the two connecting devices (rectangular-D-ring as an example) 7213, 7214.

Second, the affected arm of a user is positioned in a straight position on the said straight-arm section 1, is fastened by the arm-fixing strap 2 for fixing the straight positioned arm to the said straight-arm 1, and allows the hand of the affected arm to hold, or be fixed to, the hand-position section 3.

Third, the electrically-operated-pulling unit that of the affected side (if the affected shoulder is in left side, then, the left-side-usage type is used) is connected to the top shoulder area of the affected shoulder.

Fourth, the moving direction of the affected arm in the instant period is determined, and then, one force-applying-interface section that on the side that the arm will move toward is identified to be connected to the force-wire that come from the said electrically-operated-pulling unit in order to apply the required force toward the affected shoulder joint.

Fifth, the height of the said support-post 713 is adjusted according to the target range of the active motion to perform, in order to make the said force-wire 715 free from any friction except that from the said force-wire-supporting-point 714 during the motion of the arm. Under that the range of the moving in back and forth during therapy is not large (about 30 degrees), the mid angle of the target motion (that in back and forth) is the goal of the said adjustment on the height of the said support-post. The processes stated above are performed carefully by the motion-therapist of the user for further ensuring the following points the said force-wire 715 connecting through (the opening for the force-wire to go from the said electric machine 712, the said force-wire-supporting-point 714, and the said force-applying-interface section 4) on a plane, and which is same to the plane the moving arm makes (as schematically shown in the FIG. 4). So that the force applied by the said electrically-operated-pulling section toward the affected shoulder joint of the user can be in a correct direction.

Sixth, the magnitude of the force applied can be pre-set by the said motion-therapist of the user immediately before the active motion of the affected arm on the force-level-adjusting switch that on the said electrically-operated-pulling unit, or the said remote control device. Furthermore, the force applied is not large, being cautioned does not exceed the effective range of the dynamic force within the relevant part of the said shoulder joint. The motion-therapist that has been well trained for using the said present invention, and experienced on managing the individualized subjects in various injured-severity, recovery stages, and weight of the said arm would know how to adjust its individualized magnitude for the individualized user along the therapy sessions. Further, the said gliding section 5 can be used to ease the performance of active motion at the initial training stage, or the stage that easily fatigue. When the user has been improved, the said gliding section can be removed at the following stages.

Seventh, during the performance of the said active motion by the user, the said on/off switch is controlled by the said motion-therapist. Only after the user has well learned the timing for buttoning the on/off switch, which can be handled by the said user.

The cautions during the usage of the therapeutic active-motion apparatus that increases Joint-Electricity for improving the active motion of affected shoulder of the present invention that has included an electrically-operated-pulling section that stated in [0023] are same to those for the therapeutic active-motion apparatus that increases Joint-Electricity for improving the active motion of affected shoulder of the present invention that stated in [0021]. Thus, is not repeatedly described.

Therapeutic active-motion apparatus that increases Joint-Electricity for improving the active motion of affected shoulder of the present invention that further includes an electrically-operated-pulling section (electrically-operated-pulling-unit-that-is-situated-on-t op-of-shoulder) provides electrically-operated-pulling force toward the affected shoulder joint to actualize “a therapeutic mechanism of increasing Joint-Electricity-generation on the parts of the affected shoulder joint that relevant to the ongoing motion that following Joint-Electricity Theory, and its application theory “Using the Favorable Component for Joint-Electricity-Generation as the favorable component in motion therapy”, and consequently achieve the goals at least of immediate perception of pain-reduction, and the increase in range of active motion that is measurable. Further, the theory of the present invention and a similar structure thereof may be applied to other joints. The application of the present invention to shoulder is just an example for illustration of the present invention.

The present invention is invented based on Joint-Electricity theory and its application theory “Using the Favorable Component for Joint-Electricity Generation as the favorable component in motion therapy”. With the adequacy of the present invention, the force applied toward affected shoulder can be applied correctly and easily, and consequent the increase in the generation of Joint-Electricity that makes the increase of the range of motion and reduction in the pain. Usage of the present invention, and with a motion therapist who has been well trained in understanding and using the present invention would have the immediate result of the reduction in the shoulder pain and improvement in range of active motion that is measurable, which has been experimented and evidenced by the present inventor. Further, the present inventor has also experienced that the continuation in the usage of the present invention for weeks can improve the range of the motions in axial directions to its maximal magnitudes through using the protype of the present invention that has the same location of the said force-applying-interface sections to those included in the embodiments of the present invention. With the present invention, the therapists who are responsible for improving the shoulder problems can have a more powerful equipment to do the therapy in more of effectiveness and easiness than before.

The above provides just some preferred embodiments of the present invention and is not intended to limit the scope of the present invention. All equivalent variations and modifications that can be readily made by those having ordinary skill in the art based on the disclosure of the present invention provided in this application are considered belonging to the scope of protection of this application.

What is claimed is:

1. A therapeutic active-motion apparatus that increases Joint-Electricity for improving the active motion of affected shoulder, comprising:
a straight-arm section, which has longitudinal rigidity and a length that is sufficient to receive and support an affected arm of a user in a straight position in order to provide an effect of keeping the affected arm in the straight position during the usage of the present invention;
one or more than one arm-fixing strap, which is securely fixed to or additionally attached to the straight-arm section at least on its shoulder-end and the site corresponding to the elbow to have an effect of firmly fixing the straight-arm section to the straight positioned arm of the user;
a hand-position section, which has one-end connected to the hand-end of the said straight-arm section, and has a shape of being readily gripped by, or easily secured to, the hand of the user at the location correspond to the hand of the user on the extension of axial line of the said straight-arm section; and
one or more than one force-applying-interface section, which each is arranged on the straight-arm section at the location corresponding to one of the required directions of the motion of the affected arm, and has a shape for easily pulling the affected arm that is entirely fixed in the therapeutic active-motion apparatus of the present invention for applying a force toward the affected shoulder joint by a hand of the therapist of the said user or the contralateral hand of the user, wherein, one of the said force-applying-interface sections that on the side that the arm moves toward is selected to be used for applying the force toward the affected shoulder joint during the active motion that on the said direction.

2. The therapeutic active-motion apparatus that increases Joint-Electricity for improving active motion of affected shoulder of claim 1, further comprising a gliding section, which is connected by a gliding-section-connecting device to the distal end of the hand-position section, wherein, the said gliding-section has its distal end in low friction.

3. The therapeutic active-motion apparatus that increases Joint-Electricity for improving the active motion of affected shoulder of claim 1, further comprising an electrically-operated-pulling-section-that-on-top-of-shoulder, which is comprised of an electrically-operated-pulling unit, and a shoulder-chest-strap;
wherein, the said electrically-operated-pulling unit is comprised of:
an object-strap, which is made of a bendable material, and has a connecting device at its underside;
an electric machine, which has the function of pulling the force-wire, and is fixed to the top surface of the said object-strap, wherein, the pulling force generated by the said electric machine can be controlled by a force-level-adjusting switch, and an on/off switch, wherein, the pulling force applied during therapy can be pre-set on the said force-level-adjusting switch immediately before the active motion of the affected arm;
a support-post, which has a height that is adjustable, and is fixed to the top surface of the said object-strap on the right side, and, left side, respectively, of the side having the opening for the said force-wire to run out of the said electric machine, thus, the said electrically-operated-pulling unit has the types for right-side usage, and, left-side usage, respectively;
a force-wire-supporting-point, which allows the force-wire to go through, and change its direction, with low friction, and is comprised of a circle in smooth surface, and a thin and short post that is fixed under it, wherein, the said thin and short post is fixed to the top of the said support-post in a way that the said circle can be rotated in order to decrease any friction when the force-wire is pulled through it; and
a force-wire, which has one-end connecting to the said electric machine, and the other-end going through the said force-wire-supporting-point, and then connecting to one force-applying-interface section that on the side that the arm will move toward in the ongoing training section, and is pulled by the said electric machine to apply a force toward the affected shoulder joint; and
a shoulder-chest-strap, which can be fit to the shoulder-chest area of the affected side of the user, and has a connecting device on the area corresponding to the top area of the affected shoulder for connecting with the counterpart connecting device under the object-strap in order to position the said electrically-operated-pulling unit on the top of the affected shoulder of the user, is comprised of:
a chest-strap, which has a length-adjusting device to adjust its length for matching the circumference of the chest of the user, and has a first type connecting device set at one-end, and two second type connecting devices respectively set at the position corresponding to the central portion of the chest of the user, and, the other-end of the said chest-strap, respectively; and
a shoulder-strap, which is fixed, at an angle that is about 25 degrees, to the said chest-strap at the position corresponding to the central portion of the back of the user, and then it runs over the top of the affected shoulder and arrives at, and is connected to, the said second type connecting device that at the central portion of the chest of the said chest-strap. Further, the said shoulder-strap has a connecting device on the area corresponding to the top area of the target shoulder for connecting with the counterpart connecting device under the object-strap in order to position the said electrically-operated-pulling unit on the top of the target shoulder of the user.

4. The therapeutic active-motion apparatus that increases Joint-Electricity for improving active motion of affected shoulder of claim 3, further comprising a gliding section, which is connected by a gliding-section-connecting device to the distal end of the hand-position section, the said gliding section has a distal end in low friction.
